(19) United States
(12) Patent Application Publication
(10) Pub. No.: US 2019/0388343 A1
(43) Pub. Date: Dec. 26, 2019

(54) WATER SOLUBLE SUPPLEMENT PACKET

(71) Applicant: Paul Heynen, Calgary (CA)

(72) Inventor: Paul Heynen, Calgary (CA)

(21) Appl. No.: 16/015,514

(22) Filed: Jun. 22, 2018

(51) Int. Cl. A61K 9/00; A61K 35/741; A61K 31/198; A61K 38/16; A23L 33/175; A23L 33/135; B65D 85/808

(52) U.S. Cl. CPC A61K 9/0095; A61K 35/741; A61K 31/198; A61K 2035/115; A23L 33/175; A23L 33/135; B65D 85/808; A61K 38/16

(57) ABSTRACT

A water soluble dietary supplement packet including a water soluble membrane in a form of a packet and at least one dietary supplement in a powder form sealed within the water soluble membrane. The water soluble membrane is configured to dissolve when immersed in liquid and release the at least one dietary supplement into the liquid to be orally consumed by a user. The water soluble dietary supplement packet is formulated for enhancing physical performance when consumed. A method of preparing a dietary supplement aqueous solution is also provided.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of packages and more specifically relates to dissolvable packages.

### 2. Description of Related Art

Bodybuilding supplements are dietary supplements commonly used by those involved in bodybuilding, weightlifting, mixed martial arts, and athletics for the purpose of facilitating an increase in lean body mass. The intent is to increase muscle, increase body weight, improve athletic performance, and for some sports, to simultaneously decrease percent body fat so as to create better muscle definition. Among the most widely used are high protein drinks, branched-chain amino acids (BCAA), glutamine, arginine, essential fatty acids, creatine, HMB, and weight loss products. Supplements are sold either as pills or in a powder form. The powder form supplements may be added to water or other liquids and consumed. Often times, mixing the supplements and water as well as transporting the supplements may be difficult. A suitable solution is desired.

U.S. Pat. No. 9,643,773 to Bruce Neidle relates to a flavor pouch. The described flavor pouch has a porous covering that encloses a carrier having a flavorant. The carrier may be solid or particulate. Flavorants may be one or more of tea, coffee, rose hips, honey, royal jelly, fruit extracts, mint, sweeteners, ginger, pepper extract, exotic flavors, ethnic flavors and the like. Where the carrier comprises a plurality of beads, the beads may have randomly dissolvable encapsulation to extend the time duration of flavor release. To use the flavor pouch, it is placed in the mouth and masticated or exposed to saliva to thereby release the flavorant.

### BRIEF SUMMARY OF THE INVENTION

In view of the foregoing disadvantages inherent in the known packages art, the present disclosure provides a novel water soluble supplement packet. The general purpose of the present disclosure, which will be described subsequently in greater detail, is to provide a water soluble supplement packet to be added to water and consumed by a user.

A water soluble dietary supplement packet is disclosed herein. The water soluble dietary supplement packet includes a water soluble membrane in a form of a packet and at least one dietary supplement in a powder form sealed within the water soluble membrane. The water soluble membrane comprises a food grade plastic which is edible and water soluble. The at least one dietary supplement is prepackaged in a pre-determined amount and may include a pre-workout-supplement, glutamine, creatine, BCAAs, probiotics, protein, or other desired dietary supplement. The water soluble membrane is configured to dissolve when immersed in liquid and release the at least one dietary supplement into the liquid to be orally consumed by a user.

A method of method of preparing a dietary supplement aqueous solution is also disclosed herein. The method of method of preparing a dietary supplement aqueous solution may comprise the steps of: providing a water soluble membrane in a form of a packet and at least one dietary supplement in a powder form sealed within the water soluble membrane; immersing the water soluble membrane containing the at least one dietary supplement in a liquid to create a mixture; and consuming the mixture.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a water soluble supplement packet, constructed and operative according to the teachings of the present disclosure.

FIG. 1 is a perspective view of the water soluble dietary supplement packet during an ‘in-use’ condition, according to an embodiment of the disclosure.

FIG. 2 is a perspective view of the water soluble dietary supplement packet of FIG. 1, according to an embodiment of the present disclosure.

FIG. 3 is a perspective view of the water soluble dietary supplement packet of FIG. 1, according to an embodiment of the present disclosure.

FIG. 4 is a flow diagram illustrating a method of use for the water soluble dietary supplement packet, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to packages and more particularly to a water soluble supplement packet as used to improve the consumption of supplements by providing a portable supplement packet to be mixed with water and consumed.

Generally, the water soluble supplement packet provides a portable packet for making nutritional drinks with supplements. A user may simply add a water soluble supplement packet to water and a simple shake of the bottle mixes the required elements to be consumed by the user. The water soluble supplement packet eliminates the need for multiple bottles for supplement mixing and also eliminates the need for carrying easily confused supplements.

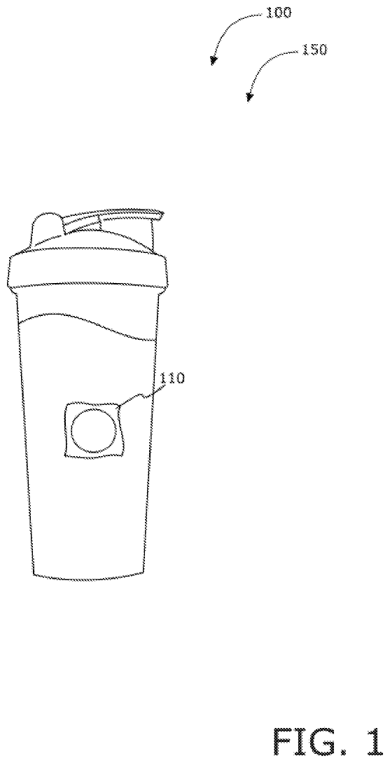

Referring now more specifically to the drawings by numerals of reference, there is shown in FIGS. 1-3, various views of a water soluble dietary supplement packet 100. FIG. 1 shows a water soluble dietary supplement packet 100 during an ‘in-use’ condition 150, according to an embodiment of the present disclosure. As illustrated, the water soluble dietary supplement packet 100 may include a water soluble membrane 110 in a form of a packet and at least one dietary supplement 120 in a powder form sealed within the water soluble membrane 110. The water soluble membrane 110 is configured to dissolve when immersed in liquid and release the at least one dietary supplement 120 into the liquid to be orally consumed by a user.

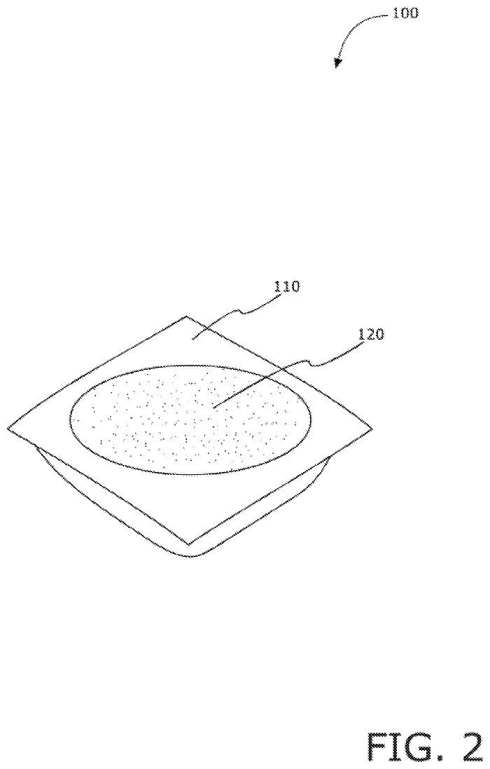

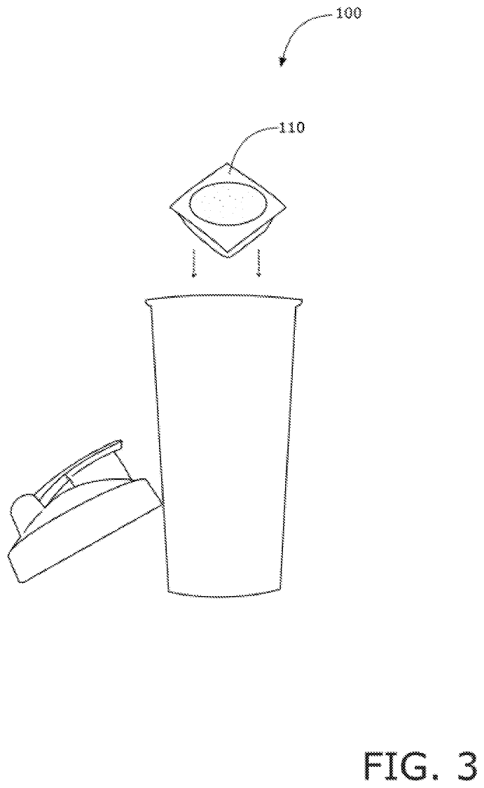

FIGS. 2-3 show views of the water soluble dietary supplement packet 100 of FIG. 1, according to an embodiment of the present disclosure. As above, the water soluble dietary supplement packet 100 may include the water soluble membrane 110 in a form of a packet configured to dissolve in a liquid such as water and release at least one dietary supplement 120 concealed within. The packet is sealable to contain the at least one dietary supplement 120 via a sealant. The water soluble dietary supplement packet is formulated for enhancing physical performance when consumed.

The water soluble membrane 110 comprises a food grade plastic which is edible and water soluble. The packet comprises a single compartment containing the at least one dietary supplement 120. The at least one dietary supplement 120 is prepackaged in a pre-determined amount and may include a pre-workout-supplement, glutamine, creatine, BCAAs, probiotics, protein, or other desired dietary supplement 120. The at least one dietary supplement 120 is water soluble. The at least one dietary supplement 120 when orally consumed by the user provides a respective effect according to the at least one dietary supplement 120 contained within the water soluble membrane 110.

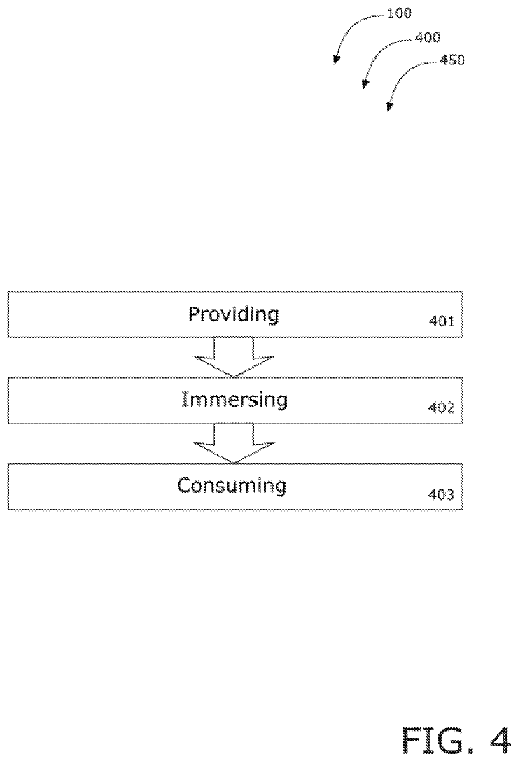

FIG. 4 is a flow diagram 450 illustrating a method of preparing a dietary supplement aqueous solution 400, according to an embodiment of the present disclosure. As illustrated, the method of preparing a dietary supplement aqueous solution 400 may include the steps of: step one 401, providing a water soluble membrane 110 in a form of a packet and at least one dietary supplement 120 in a powder form sealed within the water soluble membrane 110; step two 402, immersing the water soluble membrane 110 containing the at least one dietary supplement 120 in a liquid to create a mixture; and step three 403, consuming the mixture.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods of preparing a dietary supplement aqueous solution 400 using water soluble dietary supplement packet 100 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A water soluble dietary supplement packet comprising:
a water soluble membrane in a form of a packet; and
at least one dietary supplement in a powder form sealed within said water soluble membrane; and
wherein said water soluble membrane is configured to dissolve when immersed in liquid and release said at least one dietary supplement into said liquid to be orally consumed by a user.

2. The water soluble dietary supplement packet of claim 1, wherein said liquid is water.

3. The water soluble dietary supplement packet of claim 1, wherein said water soluble membrane comprises a food grade plastic which is edible and water soluble.

4. The water soluble dietary supplement packet of claim 1, wherein said at least one dietary supplement is selected from the group consisting of a pre-workout-supplement, glutamine, creatine, BCAAs, probiotics, and protein.

5. The water soluble dietary supplement packet of claim 1, wherein said at least one dietary supplement includes a pre-workout-supplement.

6. The water soluble dietary supplement packet of claim 1, wherein said at least one dietary supplement includes a glutamine.

7. The water soluble dietary supplement packet of claim 1, wherein said at least one dietary supplement includes a creatine.

8. The water soluble dietary supplement packet of claim 1, wherein said at least one dietary supplement includes a BCAAs.

9. The water soluble dietary supplement packet of claim 1, wherein said at least one dietary supplement includes a protein.

10. The water soluble dietary supplement packet of claim 1, wherein said at least one dietary supplement is water soluble.

11. The water soluble dietary supplement packet of claim 1, wherein said at least one dietary supplement when orally consumed by said user provides a respective effect according to said at least one dietary supplement contained within said water soluble membrane.

12. The water soluble dietary supplement packet of claim 1, is formulated for enhancing physical performance when consumed.

13. The water soluble dietary supplement packet of claim 1, wherein said packet comprises a single compartment containing said at least one dietary supplement.

14. The water soluble dietary supplement packet of claim 1, wherein said at least one dietary supplement is prepackaged in a pre-determined amount.

15. The water soluble dietary supplement packet of claim 1, wherein said packet is sealable to contain said at least one dietary supplement via a sealant.

16. A water soluble dietary supplement packet comprising:
a water soluble membrane in a form of a packet; and
at least one dietary supplement in a powder form sealed within said water soluble membrane; and
wherein said water soluble membrane is configured to dissolve when immersed in liquid and release said at least one dietary supplement into said liquid to be orally consumed by a user;
wherein said liquid is water;
wherein said water soluble membrane comprises a food grade plastic which is edible and water soluble;
wherein said at least one dietary supplement is selected from the group consisting of a pre-workout-supplement, glutamine, creatine, BCAAs, probiotics, and protein;
wherein said at least one dietary supplement when orally consumed by said user provides a respective effect according to said at least one dietary supplement contained within said water soluble membrane;
wherein said water soluble dietary supplement packet is formulated for enhancing physical performance when consumed;
wherein said packet comprises a single compartment containing said at least one dietary supplement;
wherein said at least one dietary supplement is prepackaged in a pre-determined amount; and
wherein said packet is sealable to contain said at least one dietary supplement via a sealant.

17. A method of preparing a dietary supplement aqueous solution comprising the steps of:
providing a water soluble membrane in a form of a packet and at least one dietary supplement in a powder form sealed within said water soluble membrane;
immersing said water soluble membrane containing said at least one dietary supplement in a liquid to create a mixture; and
consuming said mixture.
